(19) United States
(12) Patent Application Publication
(10) Pub. No.: US 2008/0031833 A1
(43) Pub. Date: Feb. 7, 2008

(54) Combined energy and topical composition application for regulating the condition of mammalian skin

(76) Inventors: John Erich Oblong, Loveland, OH (US); Helen Rochelle Kemp, Glendale, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/712,174

(22) Filed: Feb. 28, 2007

Related U.S. Application Data
(60) Provisional application No. 60/781,891, filed on Mar. 13, 2006.

(51) Int. Cl. A61K 31/045; A61B 18/04; A61B 18/18; A61K 31/195; A61K 31/44; A61K 38/00; A61P 17/00; A61K 8/18; A61K 31/60; A61K 31/34; A61K 31/155; A61B 18/20

(52) U.S. Cl. 424/59; 514/159; 514/17; 514/23; 514/355; 514/38; 514/474; 514/561; 514/62; 514/636; 514/729; 514/730; 514/738; 606/28; 606/33; 606/9

(57) ABSTRACT

Method for regulating the condition of mammalian skin comprising the steps of applying a first personal care composition to an area of skin where regulation is desired, wherein the first personal care composition comprises at least one skin care active selected from the group consisting of niacinamide, salicylic acid, peptides, N-acetyl glucosamine, panthenol, butylated hydroxytoluene, N-acyl amino acid compounds, hexamidine, green tea, ascorbyl glucoside, hexanediol, pentanediol, a skin lightening agent, a heat shock protein potentiator, and mixtures thereof, and delivering energy to the area of skin by contacting the skin with an energy delivery device for a treatment period of at least 2½ minutes, wherein the energy delivery device comprises a skin-contacting surface that is controllably heatable to a temperature of from 37° C. to 50° C.

### CROSS REFERENCE TO RELATED APPLICATION

This application claims the benefit of U.S. Provisional Application No. 60/781891, filed Mar. 13, 2006.

### FELD OF THE INVENTION

The present invention relates to combined applications of energy and topical compositions to mammalian skin for regulating the condition of the skin.

### BACKGROUND OF THE INVENTION

A variety of products are available to consumers to improve the condition of skin and to delay and/or prevent typical signs of aging. Such signs include, for example, fine lines, wrinkles, hyperpigmentation, sallowness, sagging, dark under-eye circles, puffy eyes, uneven skin tone, enlarged pores, diminished rate of epidermal cell turnover, and abnormal desquamation or exfoliation. For some consumers, however, the wide variety of available products and the advancements in skin care technology still fail to produce the desired results, and some feel the need to turn to more invasive medical procedures. Therefore, there is a continuing need for methods of improving the condition of skin sufficiently to avoid the need for more invasive procedures and the risks associated therewith.

### SUMMARY OF THE INVENTION

The present invention meets the aforementioned need by combining application of energy and topical compositions comprising selected skin care actives to mammalian skin. The personal care compositions of the present invention are useful for topical application and for regulating the condition of skin, and in particular, for decreasing the appearance of sagging, fine lines, wrinkles, and hyperpigmentation. Applicants believe that when applied in combination with energy, regulation of such conditions is enhanced beyond that which is achieved by application of the composition alone. The method is non-invasive, and may be performed by a consumer without the aid or supervision of a medical professional.

The following describe some non-limiting embodiments of the present invention.

According to a first embodiment of the present invention, a method for regulating the condition of mammalian skin is provided, comprising the steps of applying a first personal care composition to an area of skin where regulation is desired, wherein the first personal care composition comprises at least one skin care active selected from the group consisting of niacinamide, salicylic acid, pentapeptides, N-acetyl glucosamine, panthenol, butylated hydroxytoluene, N-acyl amino acid compounds, hexa ridine, green tea, ascorbyl glucoside, hexanediol, pentanediol, a skin lightening agent, and mixtures thereof; and delivering energy to the area of skin by contacting the skin with an energy delivery device for a treatment period of at least 2½ minutes, wherein the energy delivery device comprises a skin-contacting surface that is controllably heatable to a temperature of from 37° C. to 50° C.

According to another embodiment of the present invention, a method of reducing the appearance of fine lines and/or wrinkles in mammalian skin is provided, comprising the steps of applying a first personal care composition to an area of skin exhibiting fine lines and/or wrinkles, wherein the first personal care composition comprises at least one skin care active selected from the group consisting of niacinamide, a pentapeptide, N-acetyl glucosamine, and mixtures thereof; delivering energy to the area of skin by contacting the skin with an energy delivery device for a treatment period of at least 2½ minutes, wherein the energy delivery device comprises a skin-contacting surface that is controllably heatable to a temperature of from 37° C. to 50° C.; and applying a second personal care composition to the area of skin, wherein the second personal care composition comprises a cooling agent.

According to another embodiment of the present invention, a method of reducing the appearance of hyperpigmentation in mammalian skin is provided, comprising the steps of applying a first personal care composition to an area of skin exhibiting hyperpigmentation, wherein the first personal care composition comprises at least one skin lightening agent selected from the group consisting of N-undecylenoyl-L-phenylalanine, niacinamide, and combinations thereof; delivering energy to the area of skin by contacting the skin with an energy delivery device for a treatment period of at least 2½ minutes, wherein the energy delivery device comprises a skin-contacting surface that is controllably heatable to a temperature of from 37° C. to 50° C.; and applying a second personal care composition to the area of skin, wherein the second personal care composition comprises a cooling agent.

According to another embodiment of the present invention, an article of commerce is provided comprising a first skin care active, a second personal care composition comprising a second skin care active, an energy delivery device, and instructions that direct a user to use the first skin care composition together with an energy delivery device during a treatment period, and to use the second skin care composition between successive treatment periods.

### DETAILED DESCRIPTION OF THE INVENTION

In all embodiments of the present invention, all percentages are by weight of the total composition, unless specifically stated otherwise. All ratios are weight ratios, unless specifically stated otherwise. All ranges are inclusive and combinable. The number of significant digits conveys neither limitations on the indicated amounts nor on the accuracy of the measurements. All numerical amounts are understood to be modified by the word “about” unless otherwise specifically indicated. All measurements are understood to be made at 25° C. and at ambient conditions, where “ambient conditions” means conditions under one atmosphere of pressure and at 50% relative humidity.

It is to be understood that the steps recited in any method claims appended hereto can be performed in any order unless specified otherwise. For example, in a method claim reciting steps (a), (b) and (c), step (c) could be performed prior to or between steps (a) and (b). Furthermore, the individual steps, although recited as distinct steps, can be performed during time periods with some or complete overlap.

Herein, “regulating the condition of skin” means improving the condition of skin and/or prophylactically regulating the condition of skin, and includes, for example, protecting the tissue from ultraviolet radiation, and regulating the signs of skin aging. Herein, “improving the condition of mammalian skin” means effecting a visually and/or tactilely perceptible positive change in the appearance and feel of the tissue. Conditions that may be regulated and/or improved include, but are not limited to, one or more of the following: Reducing the appearance of wrinkles and coarse deep lines, fine lines, crevices, bumps, and large pores; thickening of skin (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by telangiectasia or spider vessels, dryness, brittleness, and graying hair.

As used herein, “signs of skin aging,” include, but are not limited to, outward visibly and tactilely perceptible manifestations, as well as any macro- or microeffects, due to skin aging. These signs may result from processes which include, but are not limited to, the development of textural discontinuities such as wrinkles and coarse deep wrinkles, fine lines, skin lines, crevices, bumps, large pores, unevenness or roughness; flaking; dryness; loss of skin elasticity; discoloration (including undereye circles); blotchiness; sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g., telangiectasia or spider vessels), and underlying tissues (e.g., fat and/or muscle), especially those proximate to the skin.

“Hyperpigmentation,” as used herein, refers to an area of skin wherein the pigmentation is greater than that of an adjacent area of skin (e.g., a pigment spot, an age spot, and the like).

Herein, “personal care composition” means compositions suitable for topical application on mammalian skin. The personal care compositions described herein may contain one or more skin care actives. “Skin care actives,” or “actives,” as used herein, means compounds that aid in regulating the condition of skin and of other mammalian skin, for example, by providing a benefit or improvement to the skin.

“Energy delivery device,” as used herein, means any device used to deliver energy to mammalian skin and/or hair. Herein, “delivery of energy,” means that the surface and/or layers of the skin are exposed to the energy emanating from the energy delivery device, where it may penetrate to desired layers of the skin, including the hair shaft and/or hair follicle.

“Continuous level,” as used herein, means that the energy delivered by the device, or energy output, remains at an essentially constant level between the time of device activation and the time of device deactivation.

“Pulsed,” as used herein, means that between the time of device activation and the time of device deactivation, the energy output varies in a predictable manner, characterized by periods of higher output (pulses) alternating with periods of lower output. The onset of pulses may be sudden or gradual. “Predictable” means that the pulse peak intensities, pulse shapes, pulse durations, and the temporal spacing between the pulses are substantially identical. The duration of the pulses and the time between pulses may vary.

“Hand-held,” as used herein, means that the device is of a weight and dimension suitable for an average adult human to comfortably hold.

Energy Delivery Devices

The method of the present invention comprises the step of delivering energy to an area of skin by contacting the area of skin with an energy delivery device. The energy delivery device typically is hand-held, and may deliver energy in a variety of forms, including but not limited to light, heat, sound (including ultrasonic waves), electrical energy, magnetic energy, electromagnetic energy (including radiofrequency waves and microwaves), mechanical energy, and combinations thereof. In one embodiment, the energy is in the form of heat energy. In another embodiment the energy is light energy. The light energy may be delivered by devices including, but not limited to, lasers, diode lasers, diode laser bars, diode laser arrays, flash lamps, intense pulsed light (IPL) sources, and combinations thereof. In one embodiment, the light energy is emitted from a laser.

The energy may be delivered in a continuous mode and/or a pulsed mode. The energy delivery device optionally may include a means for heating and/or cooling the skin prior to, simultaneously with, or after delivery of energy, and may include a means for storing compositions and for delivering one or more compositions through the device. Non-limiting examples of suitable energy delivery devices are described in U.S. Pat. No. 6,273,884. The amount of energy delivered may vary in accordance with the condition and amount of regulation of mammalian skin that is desired. In one embodiment, the energy applied to the area of skin, or “output fluence,” during a treatment period is from 1 J/cm² to 100 J/cm², where “J” means “Joules” and “cm²” means square centimeter. The energy delivery device may remain substantially stationary during the treatment period, or may be moved across the surface of the skin. For energy derived from ultraviolet light sources, the wavelength will preferably fall within the UV-A range, from 315nm to 400 nm, where “nm” means 1×10⁻⁹ meters. For energy derived from visible light sources, the wavelength will preferably range from 400 nm to 700 nm. For energy derived from infrared (IR) light sources, the wavelength will preferably range from 700 nm to 3000 nm. For pulsed light sources, the pulse length may, for example, range from 0.001 seconds to 3 seconds, with an average pulse duration of from 0.001 seconds to 1 second.

A. Light Energy Sources

Light energy includes light emitted from laser and/or non-laser light sources. The light energy may, for example, be coherent or non-coherent, monochromatic or polychromatic, and collimated, diffuse or divergent. Polychromatic light may be filtered to provide the desired wavelength or a selected band of wavelengths.

Laser light sources include solid-state lasers, gaseous lasers and combinations thereof. Non-limiting examples of solid-state laser light sources include Nd:YAG (Neodymium:Yttrium Aluminum Garnet), ruby and alexandrite. Non-limiting examples of gaseous laser sources include helium-neon, argon, and carbon dioxide. Examples of the use of suitable laser light sources are disclosed in U.S. Pat. Nos. 6,063,074 and 6,152,917, both issued to Tankovich. Additional laser light sources include, but are not limited to, diode lasers, diode laser bars, or diode laser arrays. See, for example, U.S. Pat. No. 6,273,885, issued to Koop et al.

Non-limiting examples of non-laser light sources include flashlamps, halogen lamps, light-emitting diodes (LED's), intense pulsed light (IPL) sources and combinations thereof. The wavelengths may comprise the ultraviolet, visible, near-infrared and infrared regions of the electromagnetic spectrum. Alternatively, the wavelength will be in the visible light range. Examples of suitable non-laser light sources are disclosed in U.S. Pat. Nos. 5,885,273; 6,174,325; and 6,280,438, all issued to Eckhouse et al.

Additional light energy devices suitable for use herein, include those described in the following U.S. Published Patent Applications: 2003/0216719, 2004/0167499, 2004/0167500, 2004/0167501, 2004/0167502, 2004/0167592, 2004/0176754, 2004/0176823, 2004/0176824, and 2005/0049582, and U.S. Pat. Nos.: 5,595,568; 5,735,844; 6,015,404; 6,080,146; 6,237,884; 5,669,916; 5,824,023; 5,707,403; 5,527,350; and 5,743,901. It is to be understood that alternative light energy devices are equally contemplated for use in accordance with the present invention.

B. Thermal Heat Energy Sources

Energy delivered to the skin may be in the form of thermal heat energy. The skin may be heated by broad band radiation emitted by the heat source, for example, a flash lamp. Alternatively, the heat may be generated by means of visible radiation, delivered, for example, by a high intensity lamp such as a xenon arc lamp. The heat may be generated by electrical resistivity. The heat delivery device may include a means for preventing overheating of the skin, for example, by manually or automatically distancing the apparatus from the skin, by pumping air into region surrounding the skin selected time after the flashing of the flash lamp, or by other suitable means of cooling the skin. Examples of devices that utilize heat energy are disclosed in U.S. Pat. Nos. 6,187,001; 6,245,093; and 6,635,075; and U.S. Published Patent Applications: 2001/0008974, 2003/0088298, 2004/0127962, 2005/0203596, and 2005/0288748.

C. Ultrasound Energy Sources

Energy delivered to and/or into layers of the skin may be in the form of ultrasound/ultrasonic energy. Ultrasound energy delivery may comprise higher-frequency sound waves that are greater than 40,000 Hertz (Hz), or alternatively lower frequency sound waves comprising frequencies of 40,000 Hz or less. The energy produced by the sound waves may penetrate skin, with the depth of penetration dependent upon factors including the acoustic density of the sound waves, the frequency of the sound waves, and the composition of the skin layers. The output energy generally will range from milliwatts to watts.

The delivery of the sound waves may, for example, be focused, collimated, diffuse, and combinations thereof. The delivery of the sound waves further may be continuous, pulsed, modulated, non-modulated, and combinations thereof. The ultrasound energy usually is delivered through a transducer head. When used on skin, it is usually placed in direct contact with the skin using a coupling medium, one example of which is an aqueous gel.

Examples of suitable ultrasound devices are disclosed in U.S. Published Patent Application No. 2002/0120225 Al, and U.S. Pat. Nos. 6,732,744 and 6,544,259.

D. Electromagnetic Energy Sources

Energy delivered to and/or into layers of the skin may be in the form of electromagnetic energy, including, for example, radiofrequency waves and microwaves. Exemplary electromagnetic energy devices are disclosed in the following U.S. Pat. Nos.: 6,889,090; 6,702,808; 6,662,054; 5,569,242; 5,755,753; 6,241,753; 6,430,446; 6,350,276; 5,919,219; 5,660,836; 6,413,255; 6,228,078; 5,366,443; and 6,766,202.

E. Mechanical Energy Sources

Energy delivered to the skin may be in the form of mechanical energy. Exemplary mechanical devices are disclosed in U.S. Patent Application No. 2005/0142093, published Jun. 30, 2005.

F. Other Energy Sources

Energy delivered to the skin may be generated chemically, rather than electrically. For example, devices (including e.g., patches, masks, substrates) may contain exothermic reaction technology, which upon activation can deliver thermal energy to the skin.

Other energy forms and energy sources that are known by one of ordinary skill in the art of energy may be employed by embodiments of the present invention.

Personal Care Compositions

The method of the present invention comprises the step of applying a first personal care composition and optionally a second personal care composition to an area of mammalian skin. The first and second personal care compositions may be in a variety of forms, including but not limited to lotions, creams, serums, foams, gels, sprays, ointments, masks, sticks, moisturizers, patches, powders, and/or wipes. In one embodiment, the first personal care composition is applied prior to and/or during delivery of energy. In an alternative embodiment, the second personal care composition is applied after the application of the first composition and the delivery of energy. Optionally, the method of the present invention may comprise the step of applying a third personal care composition to the skin, wherein the third composition comprises a conditioning agent. In one embodiment, the third personal care composition is applied prior to application of the first personal care composition. Preferably, the third personal care composition is applied at least 24 hours prior to the delivery of energy. In an alternative embodiment, the first personal care composition is applied twice daily and energy is delivered once daily, alternatively once weekly, and alternatively once monthly. In one embodiment, the first personal care composition is applied to the skin twice daily and energy is delivered to the skin once weekly.

The first, second and third personal care compositions may contain a variety of ingredients, non-limiting examples of which may be found in The CTFA International Cosmetic Ingredient Dictionary and Handbook, Tenth Edition (2004). In one embodiment, the first personal care composition comprises at least one skin care active selected from the group consisting of niacinamide, salicylic acid, pentapeptides, N-acetyl glucosamine, panthenol, butylated hydroxytoluene, N-acyl amino acid compounds, hexamidine, green tea, ascorbyl glucoside, hexanediol, pentanediol, a skin lightening agent, a heat shock protein potentiator, and combinations thereof. Additionally or alternatively, the first composition may comprise a sensorial evoking agent, which may be activated upon delivery of energy. In one embodiment, the second personal care composition comprises at least one skin care active selected from the group consisting of ascorbic acid, creatine, creatinine, soy extract, retinol, salicylic acid, arbutin, tranexamic acid, hydroxy acids, niacinamide, hexamidine, peptides, N-acetyl glucosamine, N-acyl amino acid compounds, green tea, ascorbyl glucoside, a sunscreen, and mixtures thereof. When the condition to be regulated includes reducing the appearance of fine lines, wrinkles, or combinations thereof, the first composition may comprise niacinamide, a pentapeptide, N-acetylglucosamine, and mixtures thereof. When the condition to be regulated includes reducing the appearance of hyperpigmentation, the first composition may comprise a skin lightening agent, non-limiting examples of which include N-undecylenoyl-L-phenylalanine, niacinamide, kojic acid, ascorbic acid, and mixtures thereof.

In other embodiments, the first and the second personal care compositions each comprise at least one additional skin care active, the combination of which may be particularly effective in regulating a given condition. Some non-limiting examples of such combinations include a first composition comprising a conditioning agent and a second composition comprising a sunscreen; a first composition comprising niacinamide and a second composition comprising a sunscreen; a first composition comprising N-acetyl glucosamine and a second composition comprising a sunscreen; and a first composition comprising a warming agent and a second composition comprising a cooling agent.

Skin Care Actives

The compositions of the present invention further may comprise at least one additional skin care active, useful for regulating and/or improving the condition of mammalian skin. Classes of suitable skin care actives include, but are not limited to vitamins, peptides and peptide derivatives, sugar amines, sunscreens, oil control agents, particulates, flavonoid compounds, hair growth regulators, antioxidants and/or anti-oxidant precursors, preservatives, phytosterols, protease inhibitors, tyrosinase inhibitors, anti-inflammatory agents, and mixtures thereof. It should be noted, however, that many skin care actives may provide more than one benefit, or operate via more than one mode of action. Therefore, classifications herein are made for the sake of convenience and are not intended to limit the active to that particular application or applications listed.

A. Vitamins

The compositions of the present invention may comprise from 0.0001% to 50%, alternatively from 0.001% to 10%, alternatively from 0.01% to 5%, and alternatively from 0.1% to 1%, of one or more vitamins. Herein, “vitamins” means vitamins, pro-vitamins, and their salts, isomers and derivatives. Non-limiting examples of suitable vitamins include: vitamin B compounds (including B1 compounds, B2 compounds, B3 compounds such as niacinamide, niacinnicotinic acid, tocopheryl nicotinate, C1-C18 nicotinic acid esters, and nicotinyl alcohol; B5 compounds, such as panthenol or “pro-B5,” pantothenic acid, pantothenyl; B6 compounds, such as pyroxidine, pyridoxal, pyridoxamine; carnitine, thiamine, riboflavin); vitamin A compounds, and all natural and/or synthetic analogs of vitamin A, including retinoids, retinol, retinyl acetate, retinyl palmitate, retinoic acid, retinaldehyde, retinyl propionate, carotenoids (pro-vitamin A), and other compounds which possess the biological activity of vitamin A; vitamin D compounds; vitamin K compounds; vitamin E compounds, or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates such as magnesium ascorbyl phosphate and sodium ascorbyl phosphate, ascorbyl glucoside, and ascorbyl sorbate; and vitamin F compounds, such as saturated and/or unsaturated fatty acids. In one embodiment, the composition comprises a vitamin selected from the group consisting of vitamin B compounds, vitamin C compounds, vitamin E compounds and mixtures thereof. Alternatively, the vitamin is selected from the group consisting of niacinamide, tocopheryl nicotinate, pyroxidine, panthenol, vitamin E, vitamin E acetate, ascorbyl phosphates, ascorbyl glucoside, and mixtures thereof.

B. Peptides and Peptide Derivatives The compositions of the present invention may comprise one or more peptides. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins, carnosine (beta-alanine-histidine), palmitoyl-lysine-threonine (pal-KT) and palmitoyl-lysine-threonine-threonine-lysine-serine (pal- KTTKS, available in a composition known as MATRIXYL®), palmitoyl-glycine-glutamine-proline-arginine (pal-GQPR, available in a composition known as RIGIN ), these three being available from Sederma, France, acetyl-glutamate-glutamate-methionine-glutamine-arginine- arginine (Ac-EEMQRR; Argireline®), and Cu-histidine-glycine-glycine (Cu-HGG, also known as IAMIN®).

The compositions may comprise from 1×10⁻⁷% to 20%, alternatively from 1×10⁻⁶% to 10%, and alternatively from 1×10⁻⁵% to 5% of the peptide.

C. Sugar Amines

The compositions of the present invention may comprise a sugar amine, also known as amino sugars, and their salts, isomers, tautomers and derivatives. Sugar amines can be synthetic or natural in origin and can be used as pure compounds or as mixtures of compounds (e.g., extracts from natural sources or mixtures of synthetic materials). For example, glucosamine is generally found in many shellfish and can also be derived from fungal sources. Sugar amine compounds useful in the present invention include, for example, N-acetyl-glucosamine, and also those described in PCT Publication WO 02/076423 and U.S. Pat. No. 6,159,485, issued to Yu, et al. In one embodiment, the composition comprises from 0.01% to 15%, alternatively from 0.1% to 10%, and alternatively from 0.5% to 5%, of the sugar amine.

D. Sunscreens

The compositions of the subject invention may comprise one or more sunscreen actives (or “sunscreens”) and/or ultraviolet light absorbers. Herein, “sunscreen” includes both sunscreen agents and physical sunblocks. Sunscreens and ultraviolet light absorbers may be organic or inorganic. Examples of suitable sunscreens and ultraviolet light absorbers are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93. Particularly suitable sunscreens include benzophenone, benzophenone-1, benzophenone-2, benzophenone-3, benzophenone-4, benzophenone-5, benzophenone-6, benzophenone-7, benzophenone-8, benzophenone-9, benzophenone-10, benzophenone- 11, benzophenone-12, benzotriazolyl dodecyl p-cresol, 3-benzylidene camphor, benzylidene camphor sulfonic acid, benzyl salicylate, bis-ethylhexyloxyphenol methoxyphenyl triazine, bornelone, bumetrizole, butyl methoxydibenzoyl-methane, butyl PABA (p-aminobenzoic acid), cinnamidopropyl-trimonium chloride, cinoxate, dea-methoxycinnamate, dibenzoxazoyl naphthalene, di-t-butyl hydroxy-benzylidene camphor, diethylamino hydroxy-benzoyl hexyl benzoate, diethylhexyl butamido triazone, diethylhexyl 2,6-naphthalate, diisopropyl ethyl cinnamate, diisopropyl methyl cinnamate, di-methoxycinnamido-propyl ethyldimonium chloride ether, dimethyl PABA ethyl cetearyldimonium tosylate, dimorpholino-pyridazinone, dimorpholino-pyridazinone, disodium bisethylphenyl triaminotriazine stilbenedisulfonate, disodium distyrylbiphenyl disulfonate, disodium phenyl dibenzimidazole tetrasulfonate, drometrizole, drometrizole trisiloxane, ethyl dihydroxypropyl PABA, ethyl diisopropyl-cinnamate, ethylhexyl bis-isopentylbenzoxazolylphenyl melamine, ethyl dimethoxybenz-ylidene dioxoimidazolidine propionate, ethylhexyl dimethyl PABA, ethylhexyl methoxy-cinnamate, ethylhexyl methoxydibenzoyl-methane, ethylhexyl salicylate, ethylhexyl triazone, ethyl methoxycinnamate, ethyl PABA, ethyl urocanate, etocrylene, 4-(2-beta-glucopyrano-siloxy) propoxy-2-hydroxybenzophenone, glyceryl ethylhexanoate dimethoxycinnamate, glyceryl PABA, glycol salicylate, hexanediol disalicylate, homosalate, isoamyl cinnamate, isoamyl p- methoxycinnamate, isopentyl trimethoxy-cinnamate trisiloxane, isopropylbenzyl salicylate, isopropyl dibenzoylmethane, isopropyl methoxy-cinnamate, kaempferia galanga root extract, menthyl anthranilate, menthyl salicylate, methoxycinnamido-propyl hydroxysultaine, methoxycinnamido-propyl laurdimonium tosylate, 4-methylbenzylidene camphor, methylene bis-benzotriazolyl tetramethylbutyl-phenol, octocrylene, octrizole, PABA, PEG-25 PABA, phenylbenzimidazole sulfonic acid, polyacrylamidomethyl benzylidene camphor, polyamide-2, polyquaternium-59, polysilicone-15, potassium methoxy-cinnamate, potassium phenyl-benzimidazole sulfonate, red petrolatum, sodium benzotriazoyl butylphenol sulfonate, sodium phenylbenz-imidazole sulfonate, sodium urocanate, TEA-phenylbenzimid-azole sulfonate, TEA-salicylate, terephthalylidene dicamphor sulfonic acid, tetrabutyl phenyl hydroxybenzoate, titanium dioxide, urocanic acid, zinc cerium oxide, zinc oxide, and mixtures thereof. In one embodiment, the composition comprises from 1% to 20%, and alternatively from 2% to 10% by weight of the composition, of the sunscreen active and/or ultraviolet light absorber. Exact amounts will vary depending upon the chosen sunscreen active and/or ultraviolet light absorber and the desired Sun Protection Factor (SPF), and are within the knowledge and judgment of one of skill in the art.

E. Oil Control Agents

The compositions of the present invention may comprise one or more compounds useful for regulating the production of skin oil, or sebum, and for improving the appearance of oily skin. Examples of suitable oil control agents include salicylic acid, dehydroacetic acid, benzoyl peroxide, vitamin B3 compounds (for example, niacinamide or tocopheryl nicotinate), their isomers, esters, salts and derivatives, and mixtures thereof. The compositions may comprise from 0.0001% to 15%, alternatively from 0.01% to 10%, alternatively from 0.1% to 5%, and alternatively from 0.2% to 2%, of an oil control agent.

F. Flavonoids

The compositions of the present invention may comprise a flavonoid. The flavonoid can be synthetic materials or obtained as extracts from natural sources, which also further may be derivatized. Examples of classes of suitable flavonoids are disclosed in U.S. Pat. No. 6,235,773, issued to Bissett, and include, but are not limited to, unsubstituted flavanones, methoxy flavanones, unsubstituted chalcones, and mixtures thereof. In one embodiment, the flavonoids are unsubstituted flavanones, unsubstituted chalcone (especially the trans-isomer), their glucosyl derivatives, and mixtures thereof. Other examples of suitable flavonoids include flavanones such as hesperidin and glucosyl hesperidin, isoflavones such as soy isoflavones, including but not limited to genistein, daidzein, quercetin, and equol, their glucosyl derivatives, 2′,4-dihydroxy chalcone, and mixtures thereof.

The compositions of the present invention may comprise from 0.01% to 20%, alternatively from 0.1% to 10%, and alternatively from 0.5% to 5% of flavonoids.

G. Cooling Agents

Compositions of the present invention may comprise one or more cooling agents. A non- exhaustive list of suitable cooling agents, includes cylic apha-keto enamines; N,2,3-trimethyl-2- isopropyl butamide; cyclohexanecarboxamide,N-ethyl-5-methyl-2-(1-methylehtyl); menthol (including, for example, natural and synthetic l-menthol); mint and peppermint. Other suitable cooling agents are disclosed in U.S. Pat. No. 6,899,901.

H. Warming Agents

Compositions of the present invention may comprise a warming agent. Exemplary warming agents include L-arginine (see, e.g., U.S. Pat. No. 5,895,658), aconite, cinnamon, evodia, sinapis, and emu oil. Other suitable warming agents are disclosed in U.S. Pat. Nos. 6,432,441; 6,899,901; and 7,005,408.

I. Heat Shock Protein Potentiators

The compositions of the present invention may comprise a heat shock protein potentiator, which may lower the temperature level of the skin-contacting surface that is otherwise required to accomplish a desired outcome. A representative, non-limiting list of heat shock protein potentiators includes heavy metals, salicylates, nonsteroidal anti-inflammatory agents, nicotine, alcohol, PPAR-gamma agonists, caffeine, and mixtures thereof.

J. Other Skin Care Actives

The compositions of the present invention further may comprise skin lightening agents, non-vitamin antioxidants and radical scavengers, minerals, preservatives, phytosterols and/or plant hormones, protease inhibitors, tyrosinase inhibitors, and anti-inflammatory agents.

Suitable skin lightening agents include, but are not limited to, kojic acid, arbutin, ascorbic acid and derivatives thereof (e.g., magnesium ascorbyl phosphate or sodium ascorbyl phosphate), extracts (e.g., mulberry extract, placental extract), N-undecylenoyl-L-phenylalanine (commercially available under the tradename SEPIWHITE® from Seppic (France), and mixtures thereof.

Suitable non-vitamin antioxidants and radical scavengers include, but are not limited to, BHT (butylated hydroxy toluene), butylated hydroxy benzoic acids, L-ergothioneine (available as THIOTANE™), tetrahydrocurcumin, cetyl pyridinium chloride, diethylhexyl syrinylidene malonate (available as OXYNEX™), 6-hydroxy-2,5 ,7, 8-tetramethylchroman-2-carboxylic acid (available as Trolox™), hexadec-8-ene-1,16-dicarboxylic acid (octadecene dioic acid; available as ARLATONE™ Dioic DCA from Uniqema), ubiquinone (co-enzyme Q10), tea extracts including green tea extract, yeast extracts or yeast culture fluid (e.g., Pitera™), gallic acid, uric acid, sorbic acid, lipoic acid, amines (e.g., N,N-diethylhydroxylamine, amino-guanidine), sulfhydryl compounds including glutathione, dihydroxy fumaric acid, lycine pidolate, arginine pilolate, nordihydroguaiaretic acid, curcumin, lysine, methionine, proline, superoxide dismutase, silymarin, grape skin/seed extracts, melanin, rosemary extracts, salts and derivatives of any of the foregoing, and combinations thereof.

Suitable minerals include zinc, manganese, magnesium, copper, iron, selenium and other mineral supplements. “Mineral” is understood to include minerals in various oxidation states, mineral complexes, salts, derivatives, and combinations thereof.

Suitable examples of plant sterols (phytosterols) and/or plant hormones include, but are not limited to, sitosterol, stigmasterol, campesterol, brassicasterol, kinetin, zeatin, and derivatives and mixtures thereof.

Suitable protease inhibitors include, but are not limited to, hexamidine, vanillin acetate, menthyl anthranilate, soybean trypsin inhibitor, Bowman-Birk inhibitor, and mixtures thereof.

Suitable tyrosinase inhibitors include, but are not limited to, sinablanca (mustard seed extract), tetrahydrocurcumin, cetyl pyridinium chloride, and mixtures thereof.

Suitable anti-inflammatory agents include, but are not limited to nonsteroidal anti-inflammatory agents (NSAIDS), including but not limited to ibuprofen, naproxen, flufenamic acid, etofenamate, aspirin, mefenamic acid, meclofenamic acid, piroxicam and felbinac; glycyrrhizic acid (also known as glycyrrhizin, glycyrrhixinic acid, and glycyrrhetinic acid glycoside), glycyrrhetenic acid, other licorice extracts; candelilla wax, bisabolol (e.g., alpha bisabolol), manjistha (extracted from plants in the genus Rubia, particularly Rubia cordifolia), and guggal (extracted from plants in the genus Commiphora, particularly Commiphora mukul), kola extract, chamomile, red clover extract, and sea whip extract, derivatives of any of the foregoing, and mixtures thereof.

Other useful skin care actives include moisturizing and/or conditioning agents, such as glycerol, petrolatum, aloe vera, allantoin, bisabolol, dipotassium glycyrrhizinate, and urea; dehydroepiandrosterone (DHEA), its analogs and derivatives; exfoliating agents, including alpha- and beta-hydroxyacids, alpha-keto acids, glycolic acid and octanoyl salicylate; desquamation actives, including zwitterionic surfactants; antimicrobial agents; anti-cellulite agents, such as caffeine, theophylline, theobromine, and aminophylline; antidandruff agents such as piroctone olamine, 3,4,4′-trichlorocarbanilide (trichlosan), triclocarban and zinc pyrithione; dimethyl aminoethanol (DMAE); creatine; (sunless) tanning agents, such as dihydroxy acetone (DHA); plant-derived materials such as resveratrol; chelators, for example, furildioxime and furilmonoxime; dialkanoyl hydroxyproline compounds; soy extracts, such as soybean milk, soybean paste, and miso salts; amino acids; topical anaesthetics, such as benzocaine, lidocaine, bupivacaine, chlorprocaine, dibucaine, etidocaine, mepivacaine, tetracaine, dyclonine, hexyl-caine, procaine, cocaine, ketamine, pramoxine, phenol; salts and derivatives of any of the foregoing; and mixtures thereof.

Dermatologically Acceptable Carrier

The compositions of the present invention may comprise from 50% to 99.9% of a dermatologically acceptable carrier. The carrier of the present invention is in the form of an emulsion. Herein, “emulsions” generally contain an aqueous phase and an oil phase. The oils may be derived from animals, plants, or petroleum, may be natural or synthetic, and may include silicone oils. Emulsion carriers include, but are not limited to, oil-in-water, water-in-oil, water-in-oil-in-water, and oil-in-water-in-silicone emulsions. In one embodiment, the dermatologically acceptable carrier comprises an oil-in-water emulsion, and alternatively, a silicone-in-water emulsion. The emulsion further may comprise a humectant, for example, glycerin and a non-ionic, cationic and/or anionic emulsifier. Suitable emulsifiers are disclosed in, for example, U.S. Pat. No. 3,755,560 issued to Dickert et al., U.S. Pat. No. 4,421,769, issued to Dixon et al., and McCutcheon's Detergents and Emulsifiers, North American Edition, pages 317-324 (1986).

Application of the Personal Care Compositions

A wide range of quantities of the compositions of the present invention can be employed to improve the condition of the skin. The quantity of the personal care composition that is applied to the skin can vary depending on the bodily location and desired benefit. Exemplary quantities include from 0.1 mg/cm² to 40 mg/cm². One useful application amount is 0.5 mg/cm² to 10 mg/cm².

A temperature change may be simultaneously induced in the skin or alternatively, in a composition applied to the surface of the skin. This temperature change is in addition to any temperature change induced by the delivered energy itself. For example, the skin may be heated prior to delivery of energy, or alternatively, the skin may be cooled before, during, and/or after delivery of energy.

Treatment Regimens

One illustrative method comprises the steps of applying a first personal care composition as described herein, to an area of skin where an improvement in appearance and/or feel is desired, and contacting the area of skin with a thermal heat device for a treatment period of at least 2/2 minutes. The thermal heat device includes a skin-contacting surface that is controllably heatable to a temperature of from 37° C. to 50° C., and preferably from 42° C. to 46° C. The personal care composition may also contain a heat shock protein potentiator. The treatment periods can be greater than 2½ minutes, such as, for example, 10 minutes or more. The thermal heat device may be placed against the skin and remain substantially stationary during the treatment period. Alternatively, the thermal heat device may be placed against the skin and moved in and/or around the targeted area of skin during the treatment period. Such movement may be continuous or non-continuous, conducted at a constant rate or a varying rate, and may have a calculated pattern or a random pattern. Further, the movement may be accomplished by manually moving the thermal heat device in and/or around the targeted skin area, be accomplished by a driven mechanism associated with the device, or a combination of the two. Manual movement may be desired by some users because it allows the device to be used in a more custom or individual fashion to meet a particular user's needs or comfort level. When the personal care composition is applied before the energy treatment, it is preferably done so within seconds or minutes of contacting the skin with the thermal heat device. For example, the energy treatment is initiated within 1 to 60 seconds, or within 1 to 10 minutes after applying the personal care composition. In some embodiments, the personal care composition is applied by or from the thermal heat device.

A second personal care composition may optionally be used in conjunction with the above-described method. The second personal care composition may be used between successive treatment periods that employ the first personal care composition and thermal heat device. The second personal care composition preferably comprises at least one skin care active not present in the first personal care composition.

Another illustrative method comprises applying a personal care composition to at least a portion of the face and/or neck, and heating the skin for a duration of at least 7 minutes via an energy deliver device during and/or following application of the personal care composition. Longer heating durations may be desired, such as, for example at least 10 minutes. The features of the personal care composition and the energy delivery device are unlimited, but preferably include at least some aspect described.

Yet another illustrative method comprises applying both a personal care composition and energy to an area of skin where an improvement in appearance and/or feel is desired. The personal care composition comprises a first active including a heat shock protein potentiator and a second active. Suitable heat shock protein potentiators include, but are not limited to, heavy metals, saicylates, nonsteroidal anti-inflammatory agents, nicotine, alcohol, PPAR-gamma agonists, caffeine, and mixtures thereof. The second active may be any of those described in the instant specification or is otherwise Yet another illustrative method comprises the steps of applying energy to an area of skin where an improvement in appearance and/or feel is desired, avoiding exposure to ultraviolet light for a period of at least 4 fours after applying the energy, and applying a personal care composition to the area of skin before, during, and/or after applying the energy. Longer periods of time wherein exposure to ultraviolet light is avoided after the energy delivery are equally contemplated, including, for example, for at least 6 hours, and for at least 8 hours.

Yet another illustrative method comprises applying a first personal care composition to skin, treating the skin with an energy delivery device within a period of time of applying the first personal care composition, and applying a second personal care composition to the skin outside the period of time of applying the first personal care composition. The period of time between applying the first personal care composition to the skin and treating the skin with an energy delivery device is preferably from 0 (meaning the composition and energy applications have at least some overlap) seconds to 30 minutes, and more preferably from about 0 seconds to 10 minutes.

Yet another illustrative method comprises the steps of applying a personal care composition to an area of skin, and directing energy to the area of skin, wherein the energy delivery device employed includes radio frequency-based energy in the absence of light-based energy.

Yet another illustrative method comprises applying a personal care composition to an area of skin where an improvement in appearance and/or feel is desired, wherein the personal care composition includes a sensorial-evoking component. The method further comprises the step of directing energy to the area of skin so that the sensorial-evoking component is activated. The sensorial-evoking component, upon activation, may provide, for example, an olfactory stimulus, a visual stimulus, a tactile stimulus, or combinations thereof. The sensorial-evoking component may be a pigment, fragrance, perfume, particulate, or other material, that is encapsulated by a coating material that melts or flows, ruptures, fractures, or otherwise releases the contained material upon interaction with the delivered energy. Further, the compositions may comprise liquid crystals that change color/appearance upon interaction with the delivered energy.

Yet another illustrative method comprises the steps applying energy to an area of skin and cooling the area of skin after applying energy to the area of skin. This method may be accomplished via an electrical device having separate energy delivering and cooling modes, that may be, for example, controlled by a user, or controlled via logic that is included with the device. This method may alternatively be accomplished with two separate devices, implements, topical compositions, substrates, or combinations thereof. For example, energy can be applied to the area of skin with an energy delivery device (such as that disclosed herein) and then a composition or substrate (e.g., a patch) loaded with a composition is applied to the area of skin, wherein the composition comprises a cooling agent. A device that cools the skin may do so via convection (forced air) or conduction (chilled surface), or via delivery of a cryogen material, for example. Exemplary cooling devices include DermaChiller4™ from Telsar Laboratories, Inc. and Cyrotherapy™ Cold Water Therapy System by Artic Ice.

### EXAMPLES

Some non-limiting examples of suitable first and second compositions include the following. Each example is suitable as either a first, second or third composition, provided that the compositions comprise the actives disclosed herein in conjunction with the respective compositions. All quantities indicate percentages by weight of the composition. Commercially available compositions suitable for use as a first composition include SK-II™ LXP Line Activating Massage Fluid and OLAY™ Regenerist Enhancing Lotion with UV Protection (SPF 15).

| | Ingredient | Ex 1 | Ex. 2 | Ex. 3 | Ex. 4 |
|---|---|---|---|---|---|
| Phase A | Water U.S.P. | QS to | QS to | QS to | QS to |
| | | 100 | 100 | 100 | 100 |
| | Disodium EDTA | 0.1 | 0.1 | 0.1 | 0.1 |
| | Allantoin | 0.2 | 0.2 | 0.2 | 0.2 |
| | Glycerin | 15 | | 10 | 20 |
| | Butylene Glycol | | 10 | | |
| | Methyl Paraben | 0.2 | 0.2 | 0.2 | 0.2 |
| | Niacinamide | 0.1 | | | |
| | Magnesium Ascorbyl | | 0.05 | | |
| | Phosphate | | | | |
| | Water-soluble | | | 2.0 | |
| | sunscreen | | | | |
| | Pal-lysine-threonine- | 0.01 | | | |
| | threonine-lysine-serine | | | | |
| | N-acetyl glucosamine | | | | 0.2 |
| Phase B | Polysorbate 40 | 2 | 2 | 2 | 3 |
| | Glyceryl Monostearate | 2 | 2 | 2 | 2 |
| | Cetyl Alcohol | 1 | 1 | 1 | 1 |
| | Stearyl Alcohol | 1 | 1 | 1 | 1 |
| | Titanium Dioxide | | 0.1 | | |
| | SAT-T-CR50¹ | | | | |
| | Vitamin E Acetate | 0.5 | 0.5 | 0.5 | 0.5 |
| | Permethyl 101 A | 5 | 5 | 3 | 3 |
| | Parsol 1789 | | | 5.0 | |
| Phase C | Sepigel 305⁵ | 1 | 1 | 1 | 1 |
| Phase D | Dow Corning 9040⁴ | 8 | | 4 | 7 |
| | KSG-21³ | | 7 | 4 | |
| | Dow Corning 245 | 2 | 3 | 4 | 2 |
| | Fragrance | 0.2 | 0.2 | | |
| | Ingredient | Ex. 5 | Ex. 6 | Ex. 7 | Ex. 8 |
| Phase A | Water U.S.P. | QS to | QS to | QS to | QS to |
| | | 100 | 100 | 100 | 100 |
| | Disodium EDTA | 0.1 | 0.1 | 0.1 | 0.1 |
| | Allantoin | 0.2 | 0.2 | 0.2 | 0.2 |
| | Glycerin | 15 | 10 | 10 | 15 |
| | Butylene Glycol | | | 5 | |
| | Methyl Paraben | 0.2 | 0.2 | 0.2 | 0.2 |
| | N-undecylenoyl-L- | | 1.0 | | |
| | phenylalanine | | | | |
| | Ascorbyl glucoside | | | 5 | |
| | Green Tea Extract | | | | 0.01 |
| | Triethanolamine | | | | 2 |
| Phase B | Polysorbate 40 | 3 | 3 | 2 | 3 |
| | Glyceryl Monostearate | 2 | 2 | 2 | 2 |
| | Cetyl Alcohol | 1 | 1 | 1 | 1 |
| | Stearyl Alcohol | 1 | 1 | 1 | 1 |
| | Farnesol | | | | |
| | Phytantriol | 5 | | | |
| | Salicylic Acid | | | 1.5 | |
| | Titanium Dioxide | | | 0.5 | |
| | SAT-T-CR50¹ | | | | |
| | PPG-15 Stearyl Ether | | | 8 | |
| | Vitamin E Acetate | | | | |
| | Permethyl 101 A | | | | 3 |
| | Parsol 1789 | | | | 2 |
| | Octocrylene | | | | 1.5 |
| | Octyl Salicylate | | | | 5 |
| Phase C | Sepigel 305⁵ | 1 | 1 | 1 | 1 |
| Phase D | Dow Corning 9040⁴ | | 7 | | 8 |
| | KSG-21³ | 5 | | 4 | |
| | Dow Corning 245 | 3 | | 2 | 3 |
| Phase E | Retinyl Propionate | | 0.2 | | |
| | Finsolv TN | | 2 | | |
| | Fragrance | 0.01 | | | |

¹Available from US Cosmetics
²Available from Roche
³Available from Shin-Etsu; 25% Dimethicone/Copolyol Crosspolymer in dimethicone
⁴12% Dimethicone/Vinyl Dimethicone crosspolymer in cyclomethicone
⁵Sepigel 305 can be purchased from Seppic and is Polyacrylamide and C13-14 isoparaffin and Laureth-7

Blend the A phase components with a suitable mixer (e.g., Tekmar model RW20DZM), heating while stirring to a temperature of 70° C. to 80° C. Separately, blend the B phase components with a suitable mixer and heat to 70-75° C. and maintain while mixing. Add Phase B to Phase A while mixing well to emulsify. When emulsion is at approximately 60° C., add Phase C while continuing to mix the emulsion. At approximately 50° C., add Phase D to the emulsion and continue mixing. At approximately 40° C., add Phase E to the emulsion Mill the emulsion using a suitable mill (Tekmar T-25) for approximately 5 minutes until a uniform product is obtained.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for regulating the condition of mammalian skin comprising the steps of:
(a) applying a first personal care composition to an area of skin where regulation is desired, wherein the first personal care composition comprises at least one skin care active selected from the group consisting of niacinamide, salicylic acid, pentapeptides, N-acetyl glucosamine, panthenol, butylated hydroxytoluene, N-acyl amino acid compounds, hexamidine, green tea, ascorbyl glucoside, hexanediol, pentanediol, a skin lightening agent, a heat shock protein potentiator, and mixtures thereof; and
(b) delivering energy to the area of skin by contacting the skin with an energy delivery device for a treatment period of at least 2½ minutes, wherein the energy delivery device comprises a skin-contacting surface that is controllably heatable to a temperature of from 37° C. to 50° C.

2. The method of claim 1, further comprising the step of applying a second personal care composition to the area of skin, wherein the second personal care composition comprises at least one skin care active selected from the group consisting of ascorbic acid, creatine, creatinine, soy extract, retinol, salicylic acid, arbutin, tranexamic acid, hydroxy acids, niacinamide, hexamidine, peptides, N-acetyl glucosamine, N-acyl amino acid compounds, green tea, ascorbyl glucoside, a sunscreen, and mixtures thereof.

3. The method of claim 2, wherein at least one skin care active in the first composition is the same as at least one skin care active in the second composition.

4. The method of claim 2, wherein the first personal care composition comprises a conditioning agent, and the second personal care composition comprises a sunscreen.

5. The method of claim 2, wherein the first personal care composition comprises niacinamide, and the second personal care composition comprises a sunscreen.

6. The method of claim 2, wherein the first personal care composition comprises N-acetyl glucosamine, and the second personal care composition comprises a sunscreen.

7. The method of claim 2, wherein the first personal care composition comprises a warming agent and the second personal care composition comprises a cooling agent.

8. The method of claim 2, wherein the first composition further comprises a sensorial evoking agent which is activated by the energy delivered by the energy delivery device.

9. The method of claim 1, wherein the energy delivery device is substantially stationary during the treatment period.

10. The method of claim 1, wherein at least a portion of the energy delivery device is moved across the surface of the skin during the treatment period.

11. The method of claim 1, wherein the energy is selected from the group consisting of heat, electromagnetic, light, radio-frequency, radio-frequency energy in the absence of light-based energy, and combinations thereof.

12. The method of claim 11, wherein the energy is heat energy.

13. The method of claim 11, wherein the energy is light energy emitted from a laser.

14. The method of claim 11, wherein the energy applied to the area of skin during the treatment period is from 1 J/cm² to 100 J/cm².

15. The method of claim 1, wherein the first personal care composition is applied to the area of skin at least twice daily.

16. The method of claim 1, wherein the energy is applied at least once per week.

17. The method of claim 1, further comprising the step of applying to the area of skin a third, pre-conditioning composition, wherein the third composition is applied at least 24 hours prior to applying energy to the skin.

18. The method of claim 1, wherein regulating the condition of skin comprises reducing the appearance of fine lines, reducing the appearance of wrinkles, reducing sagging, reducing hyperpigmentation, and combinations thereof.

19. A method of reducing the appearance of fine lines and/or wrinkles in mammalian skin, comprising the steps of:
(a) applying a first personal care composition to an area of skin exhibiting fine lines and/or wrinkles, wherein the first personal care composition comprises at least one skin care active selected from the group consisting of niacinamide, a pentapeptide, N-acetyl glucosamine, and mixtures thereof,
(b) delivering energy to the area of skin by contacting the skin with an energy delivery device for a treatment period of at least 21/2 minutes, wherein the energy delivery device comprises a skin-contacting surface that is controllably heatable to a temperature of from 37° C. to 50° C.;
(c) applying a second personal care composition to the area of skin, wherein the second personal care composition comprises a cooling agent.

20. A method of reducing the appearance of hyperpigmentation in mammalian skin, comprising the steps of:
(a) applying a first personal care composition to an area of skin exhibiting hyperpigmentation, wherein the first personal care composition comprises at least one skin lightening agent selected from the group consisting of N-undecylenoyl-L-phenylalanine, niacinamide, and combinations thereof,
(b) delivering energy to the area of skin by contacting the skin with an energy delivery device for a treatment period of at least 21/2 minutes, wherein the energy delivery device comprises a skin-contacting surface that is controllably heatable to a temperature of from 37° C. to 50° C.;
(c) applying a second personal care composition to the area of skin, wherein the second personal care composition comprises a cooling agent.

21. An article of commerce is provided comprising: a first personal care composition comprising a first skin care active; a second personal care composition comprising a second skin care active; an energy delivery device; and instructions that direct a user to use the first skin care composition together with an energy delivery device during a treatment period, and to use the second skin care composition between successive treatment periods.
